(19) United States
(12) Patent Application Publication
(10) Pub. No.: US 2009/0248143 A1
(43) Pub. Date: Oct. 1, 2009

(54) PERCUTANEOUS AORTIC VALVE ASSEMBLY

(75) Inventor: Roger J. Laham, Brookline, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: BETH ISRAEL DEACONESS MEDICAL CENTER, Boston, MA (US)

(21) Appl. No.: 12/447,016

(22) PCT Filed: Oct. 24, 2007
(86) PCT No.: PCT/US07/22510

§ 371 Date: Apr. 24, 2009

Related U.S. Application Data
(60) Provisional application No. 60/853,995, filed on Oct. 24, 2006.

(51) Int. Cl. A61F 2/82

(52) U.S. Cl. 623/1.26

(57) ABSTRACT

The present invention provides devices and methods for the treatment of cardiovascular valve diseases such as aortic stenosis. A vascular valve assembly is formed from two or more valve devices, each of which contains a valve mounted in a stent. The individual valve devices are brought to the site of the defective valve by standard percutaneous catheterization methods. Lateral expansion of the stents at the site of valve replacement produces a functioning valve assembly. Appropriate sizing and number of valve devices prevents regurgitation and migration. The assembly of two or more smaller valve devices at the site of a defective valve prevents complications due to the large size of single valve prostheses.

### CROSS REFERENCE TO RELATED APPLICATION

This application claims the priority of U.S. Provisional Application No. 60/853,995 filed on Oct. 24, 2006, the entire contents of which is incorporated herein by reference.

### BACKGROUND OF THE INVENTION

Heart valve disease can progress to the point that a valve must be repaired or replaced in order to improve the quality and/or length of life of the afflicted patient. Surgical aortic valve replacement is the only accepted treatment for calcific degenerative aortic stenosis as well as for aortic regurgitation, mitral regurgitation, and right sided valvular disease. Surgery for heart valve disease allows for the accurate replacement and repair of diseased heart valves. Traditionally, patients have had to undergo open heart surgery to treat heart valve disease. However, many patients are not candidates for surgical management due to advanced age or co-morbidities such as severe lung disease or renal failure.

There have been efforts to minimize the size of the incision but all of the other unwanted associations with open heart surgery remain. An alternative to open heart surgery is to develop methods and devices to perform heart valve repair and replacement without the need for surgery. Percutaneous treatment of valvular heart disease has only been successful in congenital stenosis, rheumatic mitral stenosis (amenable to valvuloplasty), and as a palliative or bridge procedure in adult aortic stenosis with limited success. For example, balloon aortic valvuloplasty may achieve some relief of symptoms in calcific aortic stenosis with high vascular complication rates and universal relapse and no survival benefit.

Recent efforts have focused on the development of percutaneous valving strategies with devices designed for aortic, mitral, tricuspid, and pulmonic positions for stenosis and insufficiency. For example, aortic valves mounted on a stent have been delivered antegrade or retrograde for aortic stenosis. However, the devices available are very large, thus increasing access site complication (necessitating surgery), increasing risk of damage to cardiac structures, and limiting length of the anchoring device (stent), since it has to be flexible. The current percutaneous valve designs require 21 or 24 French sheaths for delivery, resulting in the inability to deliver in patients with concomitant pulmonary vascular disease and also in a high rate of vascular complications. Some valves are available in only one size, which results in paravalvular leaks in many patients as well as in more serious conditions such as device embolization and migration.

### SUMMARY OF THE INVENTION

The present invention provides a system for the treatment of a vascular valve disease using catheter delivery. A preferred embodiment provides a valve assembly that can be used for vascular valve replacement such as the aortic valve, the mitral valve or the pulmonary valve.

In one aspect, the invention provides devices and methods for the treatment of aortic valve disease. A percutaneous aortic valve according to the invention directs the flow of blood out of the left ventricle, functionally elongates the left ventricular outflow tract (in particular the aortic annulus), limits the flow of blood back into the ventricle, and augments the blood flow through the coronary arteries. This approach yields the advantages of both a surgical and catheter-based approach, namely, safe and accurate placement of a valve between the left ventricle and the aorta as in surgery with reliable access to the aortic-ventricular junction using a catheter-based approach without the need for a surgical incision.

A preferred embodiment provides a vascular valve assembly comprising a first valve device and a second valve device. The first valve device can include a first stent and a first valve. The first stent has a delivery configuration and a deployed configuration for placement in a vascular cavity of a subject. The first valve is attached to the first stent. The second valve device can include a second stent and a second valve. The second stent has a delivery configuration and a deployed configuration for placement in the vascular cavity. The second valve is attached to the second stent. The valve assembly is formed from the lateral association or positioning of the first stent with the second stent. In another embodiment, the valve assembly further comprises a third valve device. The third valve device comprises a third stent and a third valve in which the third stent has a delivery configuration and a deployed configuration for placement in the vascular cavity. In the vascular valve assembly, the third stent can be laterally associated or positioned with the first and second stents.

The present invention also provides a method for replacing a defective vascular valve, such as an aortic valve, with a vascular valve assembly. In one embodiment, the method comprises the steps of percutaneously inserting two or more collapsed valve devices at one or more entry locations in a human or animal body, maneuvering each valve device into position within a vascular cavity to form a valve assembly. The valve assembly can be anchored to the wall of the vascular cavity by converting a stent associated with each valve device from a delivery configuration to a deployed configuration. Once maneuvered into position within the vascular cavity, the valve devices are laterally aligned. The valves of the two or more valve devices are arranged to permit flow in a common direction. In one embodiment of the method, the valve assembly replaces a defective aortic valve, and the valve assembly is positioned through the aortic annulus extending into the left ventricular outflow tract. In one embodiment of the method, each valve device is brought to the assembly site using a separate guidewire and catheter. In an embodiment of an aortic valve prosthesis, three valve devices are brought into position in the ventricular outflow tract using percutaneous catheters inserted into the left and right femoral arteries and either the left or right brachial artery.

In a first embodiment, the stents can be a self-expanding type such as nitinol, or a balloon expandable type, or a combination of both types can be used. The self-expanding type can have a locking element or device to hold the stent or valve prosthesis in the expanded position. The locking element can be tissue hooks on the exterior wall of the stent that engage the surrounding tissue, for example, or can be elements that engage oppositely positioned filaments of the stent when in the expanded position. The stent can be reversible, enabling the surgeon to collapse the stent and remove it if needed. Alternatively, each valve prosthesis can be replaced by inserting a new valve prosthesis into one or more valves that were previously placed in position.

### DESCRIPTION OF THE DRAWINGS

Other features and advantages of the invention may also be apparent from the following detailed description thereof, taken in conjunction with the accompanying drawings of which:

FIG. 1 is a schematic representation of a valve device according to the invention in a delivery configuration;

FIGS. 2A-2C are a schematic representations of embodiments of the components of a valve device; FIG. 2A is a wire cage stent, FIG. 2B is a compound leaflet valve, and FIG. 2C is a windsock valve;

FIGS. 3A-3D are diagrammatic representations of an embodiment of a method for replacing a defective aortic valve; In FIG. 3A, three guidewires from three separate percutaneous entry points are positioned within the left ventricle; In FIG. 3B, valve devices in their delivery configuration have been introduced on three separate catheters through the aortic valve and into the left ventricular outflow tract; In FIG. 3C, the balloons have been co-inflated; and in FIG. 3D the balloons, catheters, and guidewires have been removed, leaving the functioning valve assembly in position;

FIGS. 4A and 4B illustrate cross-sectional and end views of a stent and valve assembly in accordance with a preferred embodiment of the invention.

### DETAILED DESCRIPTION

The present invention relates to valving methods and devices that address many of the issues surrounding percutaneous valve replacement, including access site complications, flexibility, length of anchoring components, risk of stroke, and damage to cardiac structures. A preferred embodiment includes implanting several smaller valve devices that are assembled at the site of valve replacement to form a larger valve assembly. Each valve device contains a valve mounted within a stent. The valve assembly is a compound valve, containing two or more valve devices, each with its own valve structure. The stents are sufficiently long, flexible, and textured so that, when expanded, they apply a lateral force against the vessel wall and against each other which is sufficient to prevent significant backflow around or through the valve assembly and sufficient to prevent migration of the valve assembly. Since the valve assembly is built up at the site of valve replacement, the individual valve devices can be made small enough for routine placement by percutaneous catheter without causing significant damage to vascular or cardiac structures.

One embodiment of the invention is an aortic valve prosthesis. The valve prosthesis is formed in situ as a valve assembly composed of two or more individual valve devices. Three valve devices are preferred for replacement of an adult human aortic valve.

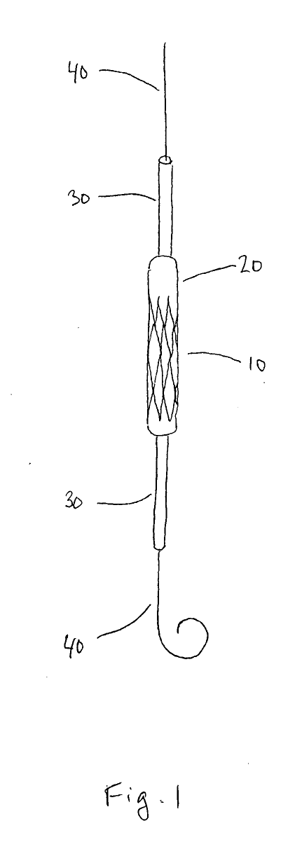

FIG. 1 shows a single valve device in delivery configuration. A stent 10 is crimped on a delivery balloon 20, which is mounted on a catheter 30, which can be delivered along a guidewire 40, using percutaneous insertion into an artery or vein by standard methods. The stent contains a valve which is not visible in FIG. 1, but which is compressed against the delivery balloon inside the stent.

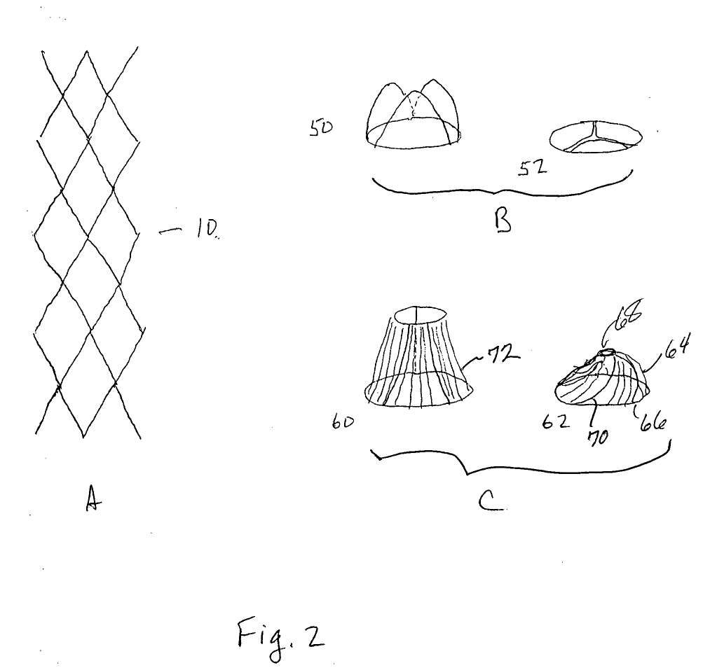

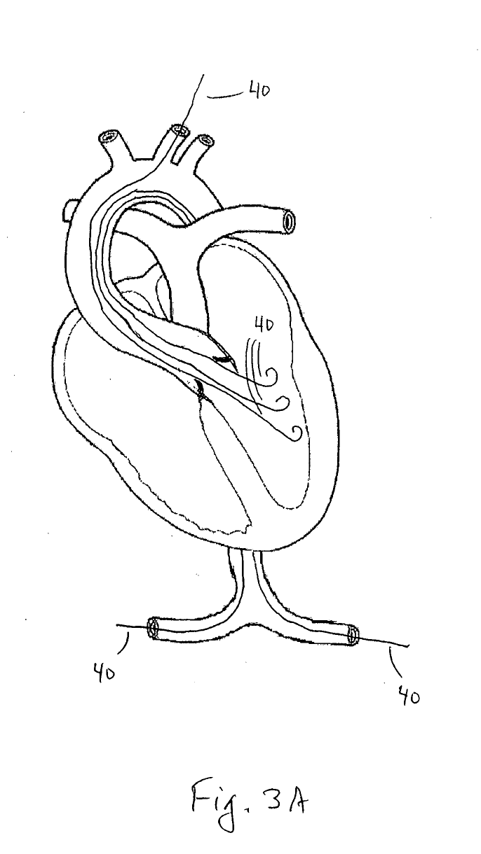

FIGS. 2A-2C show examples of components of a valve device according to the invention. One type of stent is an expandable wire cage stent 10 made of stainless steel or other suitable alloys. Many such stents are available from commercial suppliers in a variety of shapes and sizes. Preferably, the stent is longer than its expanded diameter. For example, a suitable stent for replacing an adult human aortic valve is preferably longer than 5 cm, and less than 2 cm in diameter in the deployed (inflated) configuration. A variety of natural and artificial valves are suitable for attachment to the stent to form a valve device. Mammalian valves of an appropriate size and conformation can be used. For example, neonatal porcine valves can be employed. Pericardial tissue from an animal can be fashioned into suitable valve leaflets. The valve can be a compound valve, formed from two or more individual valve leaflets. A mammolian or artificial valve composed of three valve leaflets is preferred (50, 52). Alternatively, a windsock valve can be prepared from PTFE or other materials (60, 62) as shown in FIG. 2C.

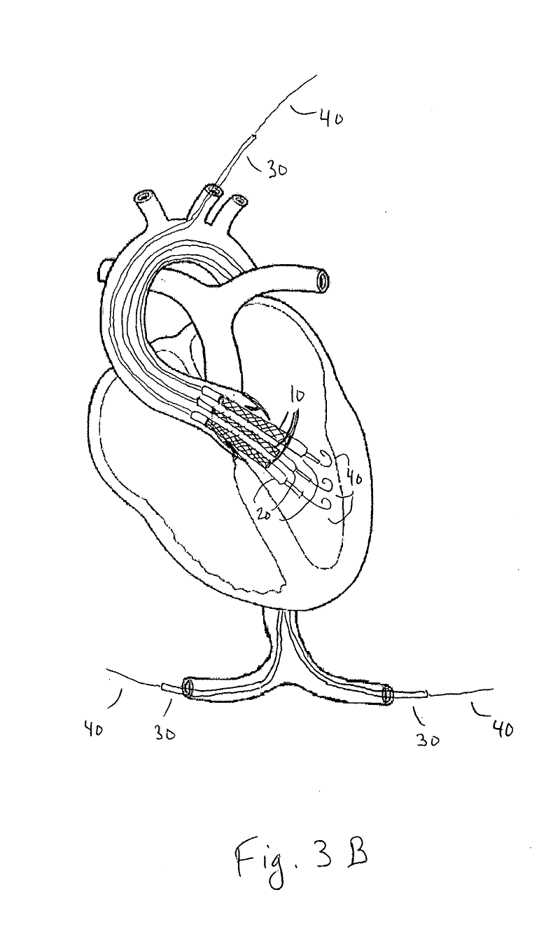

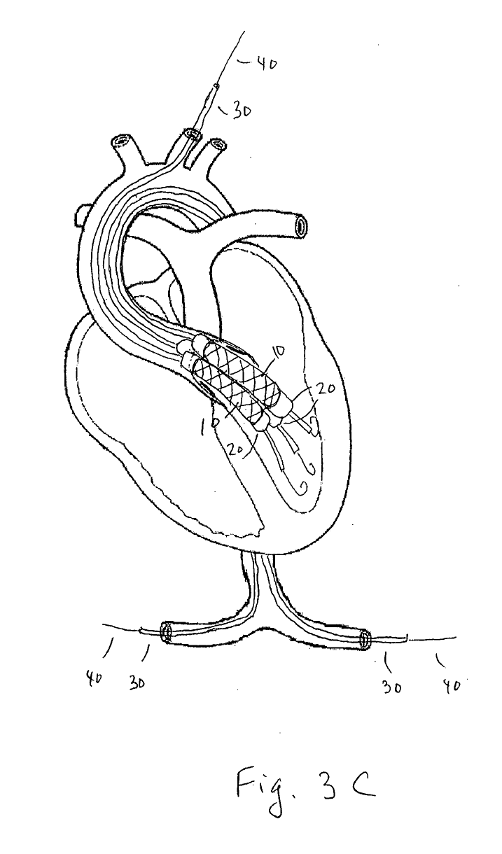

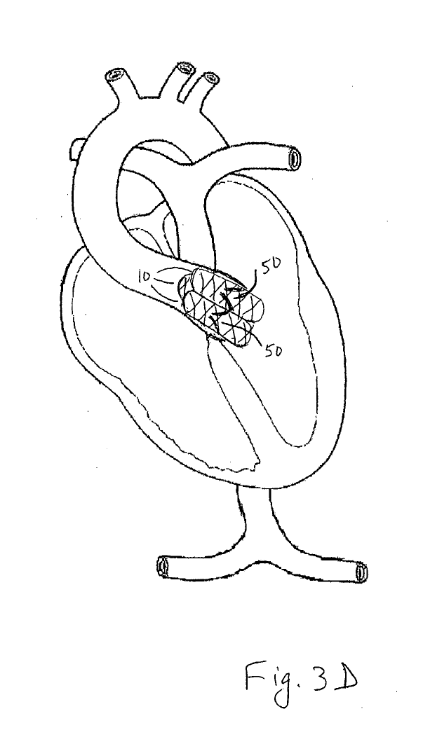

FIGS. 3A-3D illustrate a preferred embodiment of a method of replacing a defective aortic valve. Guidewires 40 are introduced by standard percutaneous catheterization. In the example shown in FIG. 3A, guidewires have been introduced into the left ventricle through the left and right femoral arteries and through the left brachial artery. FIG. 3B shows the introduction of three valve devices, one along each of the three guidewires, into position across the existing aortic valve, through the aortic annulus and extending into the left ventricular outflow tract. In this state, the wire stents 10 are still in the delivery (crimped) configuration on the balloons 20, which are mounted on catheters 30. In FIG. 3C, the three balloons have been co-inflated, approximately at the same time, and the three stents are compressed against each other laterally and against the endogenous aortic valve and the aortic annulus. Conventional echocardiography and/or fluoroscopy are used to monitor the placement of the valve devices, whose ends are preferably approximately aligned with each other. The valves within the stents are all oriented in the same direction, providing a common direction of flow. FIG. 3D depicts the functioning valve assembly following deflation of the balloons and removal of the balloons, catheters, and guidewires.

A stent can be formed by a mesh fabricated from a resilient material, such as a shape memory alloy, e.g., nitinol (nickel-titanium alloy) wire or a plastic, e.g., DELRIN. The stent can be covered with a fabric material, such as DACRON or another suitable textile, which provides structure for securing the valve relative to the stent. Shape memory (or thermal memory) is a characteristic in which a deformed part recovers to a previous shape upon heating. For example, if the stent is formed of a shape memory alloy, the stent can be inelastically deformed to a new shape, such as a reduced cross-sectional dimension, when in its low-temperature (martensitic) form. For example, a stent according to the invention can be cooled, such as by immersion in a cooling solution or fluid, and then compressed into the delivery conformation. When the stent is heated to the transformation temperature of the stent alloy, which depends on the alloy composition, the stent quickly reverts to its high-temperature (austenitic) form. The stent can be retained in the compressed condition by keeping it cooled. Alternatively, the stent can be retained in the compressed position, for example with sutures circumscribing the structure or by placing a cylindrical enclosure around the structure. The stent will then return to its original form (deployed configuration) upon removal of the retaining structure.

Alternatively, the stent can be inelastically deformable so as to require an intervening force to return the deformed stent substantially to a desired configuration. For example, a balloon catheter or spring mechanism can be employed to urge the stent and the valve located therein generally radially outward so that, after being implanted to a desired position, the stent will engage the surrounding tissue in a manner to inhibit movement relative to the surrounding tissue.

In a preferred embodiment of the invention, the stent is made of a material which is distinguishable from biological tissue to be easily visible by non invasive imaging techniques. Preferably, the stent is a stainless metal structure or a foldable plastic material, made of intercrossing filaments, preferably with a rounded and smooth linear structure. The stent is strong enough to resist the recoil phenomenon of the fibrous tissue of the diseased valve. The size of the filaments and their number are determined to give both adequate rigidity when the stent is expanded and provide a small volume when the stent is compressed. The stent can have a grate type design able to support the valve and to serve as a strong scaffold that holds open a stenosed diseased valve. When the stent is fully expanded, its intercrossing filaments push against the remains of the native stenosed valve that has been crushed aside against the vessel wall, e.g., the aortic annulus, by an inflated balloon. This produces a penetration and embeds the filaments or bars within the remains of the stenosed valve, thereby minimizing the risk of displacement.

A valve device of the invention can be compressed to a reduced cross-sectional dimension about its longitudinal axis and maintained at the reduced dimension while being implanted. Once the valve device is at a desired implantation position, and aligned with one or more other valve devices, the valve device can be returned toward its original cross-sectional dimension so as to engage remaining valve tissue, vessel wall or other surrounding tissue, and one or more adjacent valve devices at the implantation position. The engagement between the valve device and the surrounding tissue inhibits axial movement of the prosthesis relative to the tissue. In accordance with an aspect of the present invention, lateral extensions, spikes, or barbs may extend outwardly from the stent to further inhibit axial movement.

In accordance with an aspect of the invention, the stent can be covered with an outer sheath of a substantially biocompatible material. The outer sheath can have inflow and outflow ends having generally the same contour as the sidewall of the stent. The outer sheath can be formed of natural tissue pericardium (e.g., bovine, equine, porcine, etc.), another biological tissue material (e.g., collagen), or a synthetic material (e.g., Dacron). When a biological tissue is used, for example, it can be cross-linked with glutaraldehyde and detoxified, e.g. by heparin bonding.

To prevent the risk of thrombus formation and of emboli caused by clots, a substance with anti-thrombic properties, such as heparin, ticlopidine, phosphorylcholine, etc., can be employed either as a coating material for the stent, valve, and/or stent cover, or it can be incorporated into the material used for the valve and/or for the cover.

The valve portion of the valve device can be formed of any substantially biocompatible valve apparatus, for example, an animal heart valve. The valve also can be made of synthetic biocompatible material such as TEFLON, DACRON, polyethylene, or polyamide. These materials are commonly used in cardiac surgery and are quite resistant, particularly to folding movements due to the increasing systolic-diastolic movements of the valvular tissue and particularly at the junction with the frame of the implantable valve.

If the valve is formed of a natural tissue material, such as an animal heart valve, a venous valve, or a composite valve manufactured of natural tissue, the valve can be chemically fixed, for example using a fixative solution containing glutaraldehyde or another fixative, with the valve in a closed condition. The fixation process facilitates closure of the valve under back flow pressure, while remaining open during normal forward blood flow. A natural tissue valve that has been cross-linked with glutaraldehyde preferably undergoes a detoxification process such as heparin bonding (see, e.g., the NO-REACT. treatment process of Shelhigh, Inc. of Millburn, N.J.). Such treatments improve biocompatibility of the valve and mitigate calcification and thrombus formation.

One type of suitable manufactured valve is a windsock valve shown in FIG. 2C. The opening and closing of the valve can involve a helicoidal movement, which is imparted by a series of rectilinear or inclined pleats 64 formed in the tissue or fabric of the valve. The pleats 64 have an inclination from the base 66 to the upper part 68 of the valve. Pleats can be formed by folding the tissue or flexible material or by alternating thinner and thicker portions. The width and the number of pleats are variable, and depend on the type of material used. In some embodiments, the pleats are combined with inclined strengthening struts or ribs. Reinforcing pleats and/or struts, rectilinear or inclined, have the advantage of imparting a reproducible movement and, accordingly, to prevent the valve from closing to a non-structured collapse. Also, the foldable part of the valve can be reinforced by strengthening elements 70, 72 or struts to prevent an eversion of the valve towards the left ventricle during diastole. The elements function as a mechanical stop to define a second position of the valve. The valve operates as a bistable valve depending on the flow conditions. The windsock valve can be conical or generally cylindrical in shape and is attached at one end to the stent along a peripheral or circumferential edge at the base 66 or first end of the valve. The second end 59 defines an opening that can be circular or oval, for example, that opens to allow fluid flow.

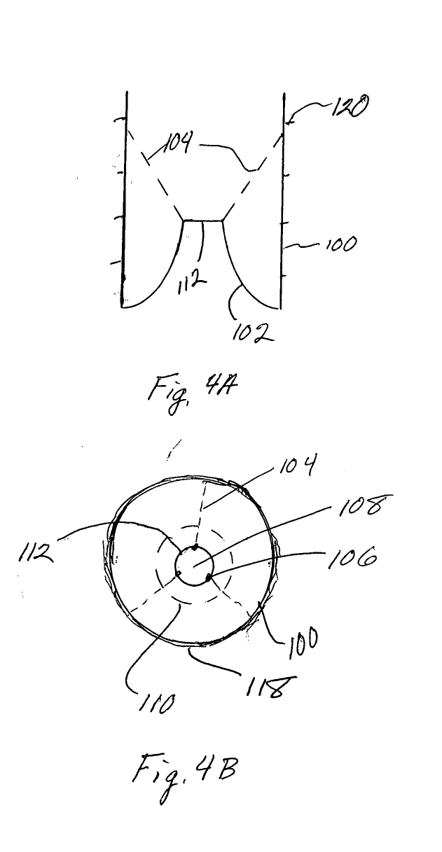

As shown in FIG. 4A, the valve 102 within a single stent 100 can have elements 104 that limit movement of the opening edge 112 of the valve. The elements 104 can be a fiber, filament or wire attached at one end to the stent wall and at a second end 106 to the edge 112 of the valve 102. As seen in the end view of FIG. 4B, the opening 108 in the valve can be a smaller diameter in a closed position and larger diameter 110 in the open position, in which more fluid can flow through the valve in one direction in the open position. The flow is substantially blocked to prevent backflow, or flow in the opposite direction. Thus, in a preferred embodiment, with an anchored windsock, the mitral valve can be treated with a self expanding stent that straddles the mitral valve annulus and contains a windsock valve whose orifice is anchored to the ventricular side of the stent. With contraction, the stent is narrowed in diameter and the windsock is prevented from leaking by closure controlled by anchors or ribs. During diastole, the stent increases in diameter and thus the valving structure is expanded and allows blood to flow unhindered into the LV from the left atrium. The stent is delivered through a transseptal approach with a wireguide placed in the LV apex and advanced to the central aorta to allow delivery of the valving apparatus.

Hooks 120 or other locking elements can be used to hold the stent to the tissue, or maintain its expanded configuration. In another embodiment, the stent or prosthesis can be coated with a material 118 such as polymer, fabric or mesh coated with a polymer that is light curable to stabilize the structure in its expanded position. A fiber optic delivery catheter can transmit UV light onto the material to lock the prosthesis into its expanded state.

The valve portion of the valve device typically exhibits structural memory. That is, when the valve is compressed into the delivery configuration, it will return substantially to its original shape (the deployed configuration) upon removal of radially inward forces, e.g., through balloon inflation. As a result, the valve is able to maintain coaptation of the leaflets even after being deformed. The valve can be fastened along a substantial portion of the stent by sewing, molding or gluing to exhibit a tightness sufficiently hermetical to prevent any regurgitation of blood around the valve. To prevent any leakage of blood, stitches are preferably numerous and very close to each other, either as separated stitches or as a continuous suture line. Also, the stitches can be made directly around the bars of the stent. Furthermore, since the valve is expanded together with the stent, the stitches, if made as a continuous suture line, are also able to expand at the same time.

Catheters and balloon catheters for use in deploying stents in the vascular system can be used. For use in the invention, a dual lumen catheter can also be used. The shaft of the balloon dilatation catheter is as small as possible, i.e., a 7 F (2.2 mm) or a 6 F (1.9 mm) size. The balloon can be mounted on the shaft. Moreover, the shaft comprises a lumen as large as possible for inflation of the balloon with diluted contrast to allow simple and fast inflation and deflation. It has also another lumen able to accept a stiff guidewire, for example 0.036 to 0.038 inches (0.97 mm), to help position the implantable valve with precision. The balloon has, for example, a 3 to 5 cm or greater length in its cylindrical part and the smallest possible diameter when completely deflated. The folded balloon preferably has at the most a section diameter of about 2.5 to 3 mm. The balloon can be made of a very thin plastic material. It can be inflated with saline containing a small amount of contrast dye in such a way as to remain visible when using X-ray visualization.

The compression of the valve devices used to form a valve assembly can be performed just prior to surgery to avoid permanent deformation of the valve structures. When the stent expands into the deployed configuration, the sidewall of the stent and any spikes or barbs associated with the stent may engage and/or be urged into surrounding tissue so as to mitigate axial movement of the valve device relative to the surrounding tissue. As a result, the valve assembly can be implanted without sutures to provide an operable valve, such as a heart valve or a venous valve.

The valve devices can be implanted in a compressed condition (the delivery configuration) by using a catheter or other structure to retain them in the compressed condition. The catheter may then be used to position the valve at a desired position, such as by utilizing a suitable imaging technology (e.g., x-ray, ultrasound, or other tomography device) or a direct line of sight. Once at the desired position, the valve devices are discharged from their retaining mechanisms (e.g., an enclosure), or their delivery balloons are inflated, so that the stents expand toward their original expanded configuration at the desired position within the vasculature, e.g., within the aortic annulus for aortic valve replacement.

Advantageously, the valve assembly may be implanted in the patient without cardiopulmonary bypass. As a result, a significant amount of time may be saved with less stress on the patient, thereby mitigating the risks of morbidity and mortality associated with conventional open-heart surgery typically employed to implant a heart valve prosthesis. Those skilled in the art will understand and appreciate that this process also may be utilized to implant a valvular prosthesis for a venous valve, such as in a patient's lower limb.

### Implantation of an Aortic Valve Prosthesis in Pigs

Three stainless steel stents each 10 mm in diameter (expanded) and 59 mm in length, were fitted with neonatal pig valves, one in each stent, to create three valve devices. The stents were crimped onto delivery balloons. Three access sites were obtained (right femoral, left femoral, and left brachial), and the stents were advanced through 8 Fr sheaths and positioned across the aortic valve area. The valves were positioned under fluoroscopic guidance as well as echocardiographic guidance.

The three valve devices were positioned over three wires (one wire from each access site). Temporary cardiac standstill was achieved with adenosine or rapid pacing. The stents were then deployed by inflating the balloons simultaneously. The balloons were then deflated, leaving the stents anchored across the aortic valve and the ascending aorta. Following a monitoring period, the pigs were sacrificed and the aorta with aortic valve was harvested and placed in 1% glutaraldehyde solution.

Three adult pigs received the aortic valve assembly. There was no immediate mortality associated with the procedure. The animals survived for 1 month without evidence of heart failure. When stents were undersized, migration was seen (in one pig). The explanted hearts demonstrated that the valves were in position and there was no evidence of thrombus formation despite lack of anticoagulation. There was no evidence of injury to other cardiac structures on gross pathological examinations.

While the invention has been described in connection with specific methods and apparatus, those skilled in the art will recognize other equivalents to the specific embodiments herein. It is to be understood that the description is by way of example and not as a limitation to the scope of the invention and these equivalents are intended to be encompassed by the claims set forth below.

1. A vascular valve assembly comprising:
a first valve device comprising a first stent and a first valve, the first stent having a delivery configuration and a deployed configuration for placement in a vascular cavity of a subject, the first valve being attached to the first stent; and
a second valve device comprising a second stent and a second valve, the second stent having a delivery configuration and a deployed configuration for placement in the vascular cavity, the second valve being attached to the second stent, and the first stent being laterally associated with the second stent to form a vascular valve assembly.

2. (canceled)

3. The valve assembly of claim 1, wherein the vascular cavity is within a heart, artery or vein.

4. The valve assembly of claim 1 wherein the first and second stents are at least 5 cm in length and less than 2 cm in diameter in the deployed configuration.

5. (canceled)

6. The valve assembly of claim 1 wherein at least one valve comprises a plurality of valve leaflets and the at least one valve is selected from the group consisting of a mammalian tissue valve, neonatal porcine valve, and pericardium and a synthetic valve.

7.-8. (canceled)

9. The valve assembly of claim 1 wherein at least one valve comprises a flexible material having a first end attached to stent and a second end having an opening that moves between an open valve position and a closed valve position.

10. (canceled)

11. The valve assembly of claim 1 wherein at least one stent comprises one or more hooks that anchor the stent in the vascular cavity.

12. A valve assembly of claim 1 wherein the ends of the stents are aligned and the valves are arranged to permit flow in a common direction, the first stent having a first sidewall contacting a second sidewall of the second stent.

13. The valve assembly of claim 1 further comprising a third valve device comprising a third stent and a third valve, the third stent having a delivery configuration and a deployed configuration for placement in the vascular cavity, the third valve attached to the third stent, and the first and second stents being laterally associated with the third stent to form a vascular valve assembly.

14. The valve assembly of claim 1 wherein the vascular valve comprises an aortic valve or a pulmonary valve.

15. (canceled)

16. The valve assembly of claim 1 wherein the vascular valve comprises an aortic valve, the stents extending through the aortic annulus into the aortic outflow tract.

17. A method of replacing a defective vascular valve with a vascular valve assembly, comprising the steps of:
percutaneously inserting two or more valve devices at an entry location in a mammalian body; and
maneuvering each valve device into position within a vascular cavity to form a valve assembly, the valve devices being laterally positioned within the vascular cavity to permit flow in a common direction.

18. The method of claim 17 further comprising anchoring the valve assembly to a wall of the vascular cavity by moving a stent associated with each valve device from a delivery configuration to a deployed configuration.

19. The method of claim 17 further comprising replacing a defective aortic valve with the valve assembly.

20. The method of claim 17 further comprising positioning the valve assembly through the aortic annulus and extending into the left ventricular outflow tract.

21. The method of claim 17 further comprising inserting three valve devices to form the valve assembly.

22. The method of claim 17 further comprising delivering each valve device using a separate guidewire and a catheter sliding over the guidewire.

23. The method of claim 22 further comprising inserting a first catheter into a left femoral artery and a second catheter into a right femoral artery and inserting a third catheter into a brachial artery.

24. (canceled)

25. The method of claim 17 further comprising inserting a first valve through a first percutaneous location and inserting a second valve through a second percutaneous location different from the first location.

26. (canceled)

27. A valve prosthesis comprising:
a valve device including a prosthesis and a valve, the prosthesis having a delivery configuration and a deployed configuration for lateral placement of a plurality of said valve devices in a vascular cavity of a subject, the valve being attached to the prosthesis at a first end of the valve and have a variable diameter opening at a second end of the valve, the valve comprising a flexible material in which the opening moves between an open valve position and a closed valve position.

28. The valve prosthesis of claim 27 wherein the vascular cavity is within a heart, an artery or a vein.

29. (canceled)

30. The valve prosthesis of claim 27 wherein the stent is at least 5 cm in length and is less than 2 cm in diameter in the deployed configuration.

31. (canceled)

32. The valve prosthesis of claim 27 wherein the valve is selected from the group consisting of a mammalian tissue valve and a synthetic valve.

33.-36. (canceled)

37. The valve prosthesis of claim 27 wherein the stent comprises one or more hooks that facilitate anchoring in the vascular cavity.

38.-40. (canceled)

41. A vascular valve delivery assembly comprising:
a first valve device comprising a first stent and a first valve, the first stent having a delivery configuration and a deployed configuration for placement in a vascular cavity of a subject, the first valve being attached to the first stent;
a first catheter for delivering the first valve device at a first percutaneous location;
a second valve device comprising a second stent and a second valve, the second stent having a delivery configuration and a deployed configuration for lateral placement with the first valve device in the vascular cavity, the second valve being attached to the second stent; and
a second catheter for delivery of the second valve device at a second percutaneous location such that the first stent is associated with the second stent to form a vascular valve assembly.

42. (canceled)

43. The valve delivery assembly of claim 41 wherein the vascular cavity is within the heart.

44.-50. (canceled)

51. The valve delivery assembly of claim 41 wherein at least one of stents comprise one or more locking elements that facilitate anchoring.

52.-56. (canceled)

57. The valve delivery assembly of claim 41 wherein the first catheter delivery location is a left femoral artery and the second delivery location is a right femoral artery.

58. The valve delivery assembly of claim 41 wherein a third catheter delivery location is a brachial artery.

59.-65. (canceled)
